(19) United States
(12) Patent Application Publication
(10) Pub. No.: US 2020/0013029 A1
(43) Pub. Date: Jan. 9, 2020

(54) Systems and Methods for Aging, Disability and Palliative (ADP) Care Utility Savings and Exchange

(71) Applicant: FEI.com, Inc. dba FEI Systems, Columbia, MD (US)

(72) Inventors: Jiao Z. Gu, Lake Mary, FL (US); Steven Reynolds, Port St. Lucie, FL (US); Chirag Bhatt, Silver Spring, MD (US)

(21) Appl. No.: 16/412,364

(22) Filed: May 14, 2019

Related U.S. Application Data
(60) Provisional application No. 62/695,012, filed on Jul. 7, 2018.

(51) Int. Cl. G06Q 20/10; G06Q 20/40; G16H 10/60; G06Q 10/10

(52) U.S. Cl. CPC G06Q 20/10; G06Q 20/4014; G06Q 40/08; G06Q 10/10; G16H 10/60

(57) ABSTRACT

A method and system to acquire and make payments for Aging, Disability and Palliative (ADP) support services utilizing normalized digital tokens. The system and method provides for a needs assessment and determination of a consumer's ADP services needs for their potential lifespan; normalization of the digital assets based on attributes associated with the tokens such as service level, service type, time and location; and for claims and payment mechanisms to support multiple payment and payer sources depending on eligible and authorization. The system includes an ADP balance sheet to maintain a consumer's assets, including digital tokens, and corresponding liabilities; and an exchange for the acquisition and trading of the digital assets and the ADP support services amongst the participants through various mechanisms. The system and method also provides for a blockchain network to enhance tracking of the assets for the various transactions.

### CROSS-REFERENCE TO RELATED APPLICATIONS

The present application claims the benefit, under 35 U.S.C. § 119(e), of U.S. Provisional Patent Application Ser. No. 62/695,012 filed on Jul. 7, 2018, entitled “Methods and Systems for Aging, Disability and Palliative (ADP) Care Utility Savings and Exchange,” which is incorporated herein by reference in its entirety.

### BACKGROUND OF THE INVENTION

### Field of the Invention

The present invention relates generally to methods and systems that facilitate acquisition of and payment for aging, disability and palliative (ADP) support services via normalized digital tokens and a shared economy.

### Description of Related Art

The aging of the world's population is driving a deep-seated problem of the utilization of resources, and the strain on those resources. The number of older people across the world is growing at an unprecedented rate. In the United States and throughout the world, people who are 65 years old and older comprise the fastest growing segment of the population. Worldwide, those aged 65 years old or above are estimated to increase from a recent rate of 8.5 percent of our population (617 million) to nearly 17 percent (1.6 billion) by 2050. An Aging World: 2015, International Population Reports, WanHe, Daniel Goodkind, Paul Kowal, US Census Bureau, (March 2016). At the same time, global life expectancy at birth is projected to increase by almost eight years, climbing to 76 years in 2050.” Those individuals that are aged 80 years and older world-wide are estimated to more than triple between 2015 and 2050. The oldest population in some Asian and Latin American countries is predicted to quadruple by 2050. By 2060, it is estimated that there will be 98 million older Americans, up from 48 million in 2015. They will comprise one in four Americans, and almost 20 million will be over the age of 85. Profile America Facts for Features: Older Americans Month: May 2017, US Census Bureau (Mar. 27, 2017).

This shift in the world's population is reflected in the decline of the potential support ratio (PSR)—meaning the ratio of the working age population, 15 to 64 years of age, to the population 65 years and older. Number of Workers per Retiree Declines Worldwide, Joseph Chamie, Dec. 22, 2015, Yale Global and the MacMillan Center. Lower PSRs will place increasing stress on economies, as fewer workers must support increasing numbers of elderly citizens. Changes in the ratios across the world will be significant. In 1950, the global potential support ratio was about 12 people of working age per one person aged 65 years or older. Today, the world PSR has declined to eight. By the year 2050, the world PSR is projected to decline to four. By 2050, some countries will have PSRs under two.

In most of the world, much of the care for our older population is provided by friends and relatives. There are currently various government (e.g. Medicare, state Medicaid directly or Managed Care Organizations) plans that assist consumers with some costs during their retirement years. However, the majority of long term services and support is currently provided by unpaid caregivers. And currently, some insurance programs will not pay for personal caregivers. As the PSR declines, there will not be enough friends and relatives to provide such care. More and more care will have to be provided by paid workers. Personal-care aides and home-care aides are one of the fastest growing occupational categories in the United States. The Sourcebook, Essentials of Health Policy, Chapter 10—Long-Term Services and Supports (Aug. 31, 2017).

Across the world, most government programs and commercial agents of the government continue to operate disparate programs that do not address individuals holistically. More importantly, they do not allow current populations to put in place methodologies today that will support them and their peers as they age in the future. Without change today, generations to come will face unrelenting cost pressures that will consume much of the economic resources of most countries just to allow aging populations to live out their lives.

Some methods have been developed to help individuals with selected portions of their planning for their retirement years and with acquiring and paying for ADP support services. For example, U.S. Pat. No. 7,647,261 sets forth a method for retirement income planning. This method includes receiving data in connection with an individual's projected income and the individual's projected expenses that are used to determine a projected income stream and the probability that such individual will achieve his desired income stream. The method then describes the purchase of annuities from various sources to fill any deficiency in the income stream, if the individual can afford such annuities. U.S. Pat. No. 7,627,512 sets forth a method that utilizes asset and liabilities data of an individual to forecast financial variables and to visually display the results of the financial model.

As another example, U.S. patent application Ser. No. 15/223,458 sets forth a method for assessing an individual's current assets and liabilities and attempts to address optimized resource usage programs and to model selected location preferences for retirement. U.S. patent application Ser. No. 11/504,451 sets forth a method for evaluating availability and eligibility of benefits for individuals. U.S. patent application Ser. No. 13/803,363 sets forth a method for matching consumers to providers of healthcare goods and services and facilitating payment. U.S. patent application Ser. No. 15/186,035 sets forth a method for managing payout splits to parties to a shared economy transaction including receiving the customer request, transmitting the customer request to at least one service provider, and receiving notice of completion of the transaction.

U.S. patent application Ser. No. 14/929,287 sets forth a method for purchase a number of care hours in advance and describes changing one's model into care hours and depositing those hours into one's care account.

Accordingly, what is needed is a method and system for improvement and innovation of current plans via unconventional steps whereby individuals can acquire and continue to acquire sufficient, alternative assets to pay for the ADP support services needed during their increasingly longer lifetimes, such assets to be monetized in token form. What is further needed is a method and system to normalize the value of such token based upon prescribed rules and other factors such as the types of ADP support services, and the time and location for the provision of such services. What is further needed is a method and system for procuring such ADP support services via a shared economy model to access these limited ADP support services.

### BRIEF SUMMARY OF THE INVENTION

A system for acquisition and payment of aging, disability, and palliative (ADP) services, using at least one computing device is provided is described in this application. The computing device is configured to implement a method comprising several steps. In one step the computing device is configured to register at least one consumer by storing said consumer's consumer information, including said consumer's name and other identifying information and creating a consumer account for said consumer from said consumer information. In another step, the computing device receives from said consumer a consumer itemization of said consumer's assets and liabilities. The computing device then creates and stores a consumer ADP balance sheet containing said consumer itemization in the consumer account. The system creates estimates of needs and liabilities of said consumer over a future period of time and stores the estimates in the consumer ADP balance sheet. The system, in another step, acquires a digital token for purchasing ADP services and updates the consumer ADP balance sheet to reflect the digital token acquired.

The system also provides that the computing device registers at least one service provider by storing said service provider's provider information, including said service provider's name and other identifying information in a provider account. The system creates and stores a provider ADP balance sheet of said service provider in said provider account. In another step, the system selects a service provider and estimates a value for ADP services to be provided to the consumer. The system procures ADP services from a selected service provider and makes payment for provided ADP services in an amount of digital tokens from the consumer account. The system then updates the consumer ADP balance sheet and the provider ADP balance sheet to reflect the payment.

In some embodiments, the digital tokens, the consumer ADP balance sheet, and the provider ADP balance sheet are stored in a blockchain. In further embodiments, the digital tokens are acquired by said consumer performing volunteer work in exchange for the digital tokens. The consumer may also earn digital tokens in accordance with actions of said consumer in response to requests via the ADP services exchange. In another embodiment at least one third party makes a donation of digital tokens to the consumer account. In further embodiments, the ADP services exchange transmits an alert to said consumer upon a change in the amount of digital tokens in the consumer account.

In an alternative embodiment, the computing device is further configured to register at least one payer by entering said payer information, including the payer name and other identifying information in a payer account. The computing device also creates and stores a payer ADP balance sheet in said payer account. The system further receives a request from at least one consumer to procure digital tokens from at least one payer. The computing device, in another embodiment, transfers digital tokens from the payer account to the consumer account; and updates the consumer ADP balance sheet and payer ADP balance sheet to reflect the transfer.

In one exemplary embodiment, the amount of digital tokens required for the payment for the ADP services is normalized based upon service type, service level, location and time. The normalization of the amount of digital tokens, in some embodiments, is performed via an artificial intelligence engine. In another embodiment, at least one service provider is recommended to said at least one consumer via the ADP services exchange for the service desired by said consumer, based upon said consumer's past experience with service providers, reviews and ratings by other consumers, location, and availability.

A method for acquisition and payment of aging, disability, and palliative (ADP) services is further described herein. In one step of the method includes registering, via a computing device, at least one consumer by storing said consumer's consumer information, including said consumer's name and other identifying information and creating a consumer account from said consumer information. Another step includes receiving, via the computing device, from said consumer a consumer itemization of the consumer's assets and liabilities. Yet a further step is creating and storing a consumer ADP balance sheet containing said consumer itemization in the consumer account. The method also provides for creating estimates of needs and liabilities of said consumer over a future period of time and storing the estimates in the consumer ADP balance sheet; acquiring a digital token for purchase of ADP services; updating the consumer ADP balance sheet to include said digital token acquired; registering, via a computing device, at least one service provider by storing said service provider's provider information, including name and other identifying information, in a service provider account; and creating and storing a provider ADP balance sheet in said provider account.

The method includes selecting at least one service provider and estimating a value for ADP services to be provided to said consumer; procuring ADP services from at least one service provider; making payment for provided ADP services in an amount of digital tokens from the consumer account; and updating the consumer ADP balance sheet and provider ADP balance sheet to reflect the payment.

Some additional embodiments provide a method wherein the consumer accesses and registers via ADP services exchange via an application installed on said consumer's computing device. In further embodiments, the digital tokens, the consumer ADP balance sheet, and the provider ADP balance sheet are stored in a blockchain. In another embodiment, the consumer acquires said digital tokens for performing volunteer work or a third party makes a donation of digital tokens to the consumer account. In further embodiments, the ADP services exchange grants digital tokens to said consumer account.

In yet further embodiments the method includes the steps of registering, via a computing device, at least one a payer by storing said payer information, including the payer's name and other identifying information and creating a payer account from said payer's information; creating and storing an ADP balance sheet of said payer in said payer account; receiving, via a computer device, request from the consumer to procure digital tokens from said payer; receiving digital tokens from the payer; and updating the payer ADP balance sheet and the consumer payer sheet to reflect an exchange of digital tokens.

In a further embodiment, the ADP services exchange transmits an alert to said consumer upon a change in the amount of digital tokens in said consumer account. In yet a further embodiment, the amount of digital tokens required for the payment for the ADP services is normalized based upon service type, service level, location and time. The normalization of the amount of digital tokens, in some embodiments, is performed via an artificial intelligence engine. In another embodiment, at least one service provider is recommended to the consumer via the ADP services exchange for a service desired by said consumer, based upon said consumer's past experience with service providers, reviews and ratings by other consumers, location, and availability.

Each of these features is described with details in the detailed description of the invention in subsequent section.

### BRIEF DESCRIPTION OF THE DRAWINGS

Other objects and advantages of the invention will become apparent from a study of the following specification when viewed in light of the accompanying drawings, in which:

FIG. 1 is a diagram of ADP support services workflow for creating and managing digital assets in various forms to establish an ADP balance sheet.

FIG. 2 is a diagram of ADP support services workflow for intelligent service planning for service and provider recommendation that provide best value.

FIG. 3 is a diagram of ADP support services workflow for the service provision for the different types of services.

FIG. 4 is a diagram of ADP support services workflow for the billing process related to different types of payment sources.

FIG. 5 is a diagram of the ADP support services workflow interfaces.

FIG. 6 is a diagram of various sources that contribute towards building ADP assets—via token or reward form.

FIG. 7 is a diagram of attribute based normalization of specialized balance sheets including assets and liabilities.

FIG. 8 shows an example of a hybrid balance sheet with various assets, liabilities and remittance methods.

FIG. 9 shows a computing device for use with the invention.

### DETAILED DESCRIPTION OF THE INVENTION

Referring to the drawings generally, the methods and systems depicted in the drawings can be implemented by use of an online computer software application operated from a computing device.

The systems and methods described here allow a consumer to acquire and make payment for aging, disability and palliative (ADP) support services using digital tokens having a normalized value. The methods and systems can be implemented by use of an online software application. This system and method provides for a needs assessment for a full life or a specified time period to capture health, social, financial and environment needs information. The system and method further provides for prediction and planning for future ADP long-term care needs, recommend services and providers, analysis and_provision of the best value plan.

The system and method also provide for an integrated network to connect consumers (retirees/beneficiaries), providers and payers including authorized representatives or care managers to operate on behalf of consumers. The system and methods further provide for coordination of the personal care, housing, transportation, meals and other personal services. The system and method also provides for the acquisition of assets in token form to be used to make payment for the ADP support services. The system and methods provide for an ADP balance sheet to maintain a consumer's assets (including these digital tokens) and corresponding liabilities.

The systems and methods provide for normalization of the digital tokens based on attributes and rules associated with the digital tokens such as service level, time and location. The system and methods provide for an exchange for the acquisition and trading of the digital assets and the ADP support services amongst the participants through various mechanisms. The system and methods provide for claims and payment mechanisms to support multiple payment and payer sources depending on eligibility and authorization. The system and methods provide for a blockchain network to enhance tracking of the assets for the various transactions.

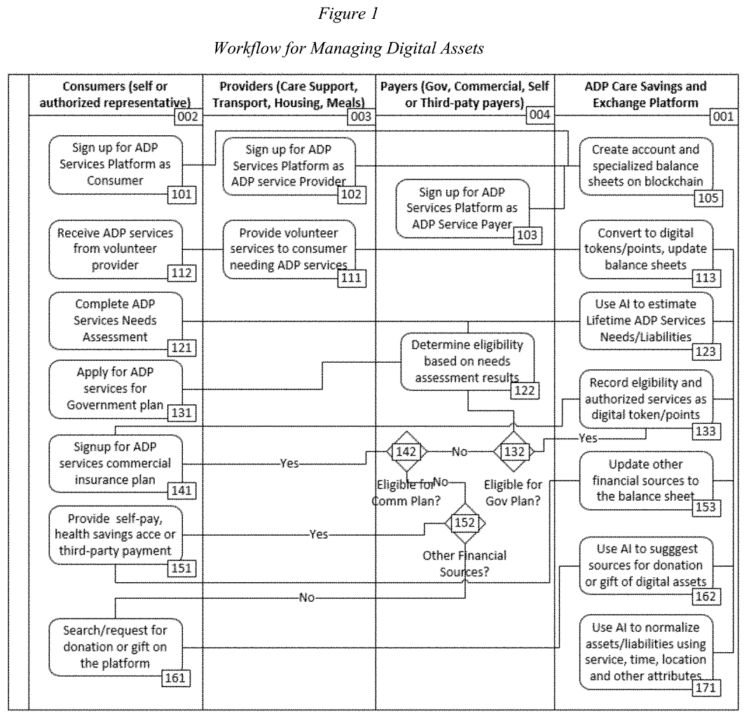

Referring to FIG. 1, the overall workflow for creating and managing digital assets through the ADP services exchange 001 is depicted. The depiction includes three types of participants: consumers 002, providers 003 and payers 004. Participants can sign up to utilize the ADP services exchange 001 through workflow steps for consumer 101, providers 102 and payers 103, resulting in accounts created with specialized balance sheets available on the exchange and within the blockchain 105. The solution supports the ability of a volunteer service provider to provide services 111 which will be received by the consumer who is in need of ADP services 112. The ADP services exchange 001 converts this volunteer effort to equivalent digital assets (cryptographic digital tokens or reward points) and updates the specialized ADP balance sheets 113 (i.e., a consumer ADP balance sheet, a provider ADP balance sheet, and a payer ADP balance sheet) for the consumer and provider involved in the volunteer service transaction. As referred to in this application, a “digital token” refers to a utility token utilized as a medium of exchange that gives one access to the value created or exchanged, and at times, the tokens are in the blockchain network. Participants in the ADP services exchange will acquire digital tokens as representatives for their assets. Digital tokens are also used to exchange value between the two participants, e.g. a provider compensated by digital tokens for the service provided to a consumer. It is contemplated that digital tokens can have any assigned value and, when referring to a digital token, it is contemplated that fractions of such digital tokens come within the definition of the digital token. This allows for earning and storing of digital assets that volunteer providers can avail themselves of as consumers at later time for any ADP services at any location. The consumer, provider, payer and other participants may be provided an application to be installed on their remote computer device or mobile device to allow access to the ADP services exchange.

The consumer completes the ADP needs assessment 121 that is in turn processed by the ADP services exchange 001 to estimate ADP services needs/liabilities for a life-time or a specified time period 123. The consumer provides age, desired retirement age, location, ADP preferences and other information. These liabilities become a major component of the ADP balance sheets. The needs assessment is also used to determine functional eligibility 122 for government (e.g. Medicare, state Medicaid directly or Managed Care Organizations) plans 132. If consumer is found to be eligible, then eligibility and authorized level of services are recorded as digital assets 133 that may be used to satisfy ADP needs/liabilities. If the consumer is not eligible for any government plan due to age, income, physical assets or other criteria, then the same consumer data is used to check eligibility for commercial insurance plan 142. If eligible, the consumer can then sign up for the insurance plan 141 which is also recorded as digital assets 133.

When a consumer is not eligible for either a government plan or commercial plan, then the ADP services exchange checks if the consumer has other financial sources 152 including, for example, self-pay, health savings account, and third-party payment, that the consumer can provide 151. Any sources uncovered can be updated within the ADP services exchange as digital assets on the ADP balance sheet 153. When the consumer has no other financial sources, the exchange allows for a search or request for donations or gifts available 161. This request is processed to suggest sources of donation or gift in the form of digital assets (digital tokens or points) 162. All of the digital assets and liabilities described above are again processed by the exchange to normalize the value using various attributes i.e. service type, time, location and provider settings 171 (details provided later in this section).

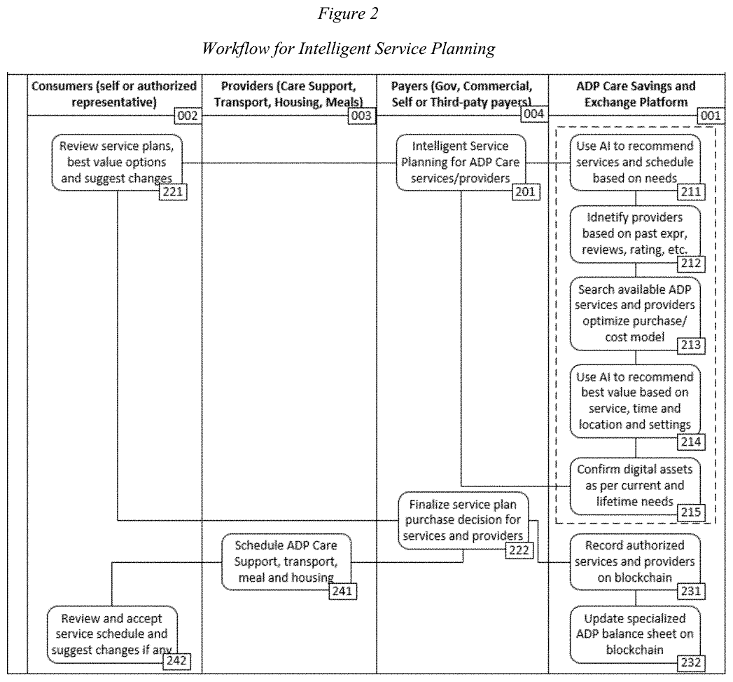

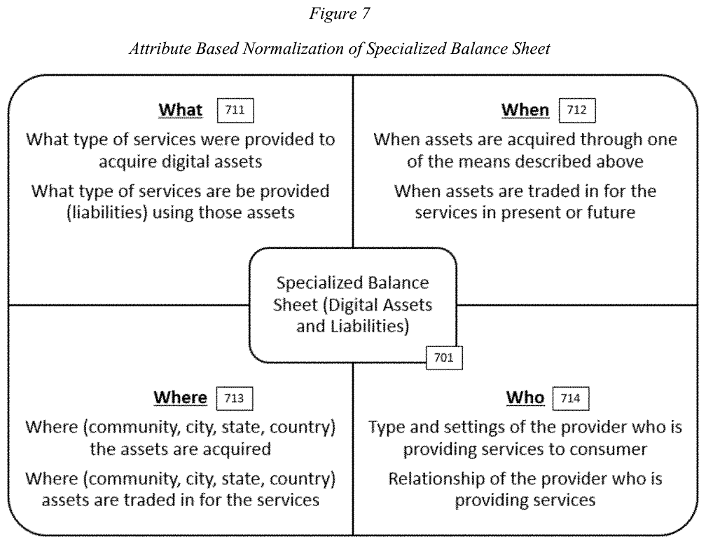

Referring to FIG. 2, the overall workflow for intelligent service planning through the ADP services exchange 001 is depicted with primary involvement from payers 004 and consumers 002. Intelligent service planning is initiated by the payers to identify ADP services and providers aligned with the care needs 201. Note that the payers include government or commercial plan designated support coordinators as well as consumers themselves for self-pay or their guardians or sponsors for the third-party private pay 004. The exchange 001 provides recommendations of the services and corresponding schedule based on the needs 211. The service providers for those services are also recommended and identified by the platform based on the consumer's past experience with the providers as well as reviews and ratings 212 by all consumers that have received services from that provider, location, availability, and other factors. The exchange then searches all available ADP services and providers to present an optimized purchase/cost model 213. Lastly the ADP services exchange 001 processes the normalized value of the available digital assets (as described in further detail below and in FIG. 7) and needs/liabilities based on associated attributes i.e. service type, time, location and settings, and makes best value recommendations including alternate plans to satisfy the same ADP needs with lower cost and/or higher quality 214. The exchange 001 confirms the availability of digital assets as stored on the consumer ADP balance sheet 113 as per the current and lifetime needs 215.

The above described service planning workflow is an iterative process until the payer determines an appropriate plan with services and providers offering the overall best value. This plan is reviewed by the consumer who in-turn suggests changes if necessary 221. It should be noted that this step is not relevant when payer will also be the consumer in a self-pay scenario. The service plan is then finalized to make the purchase decision by the payer 222. The services and authorized providers on the plan are recorded on the blockchain for service verification at a later time 231. The platform also updates the digital assets and liabilities on the consumer ADP balance sheet 113 on the blockchain 105.

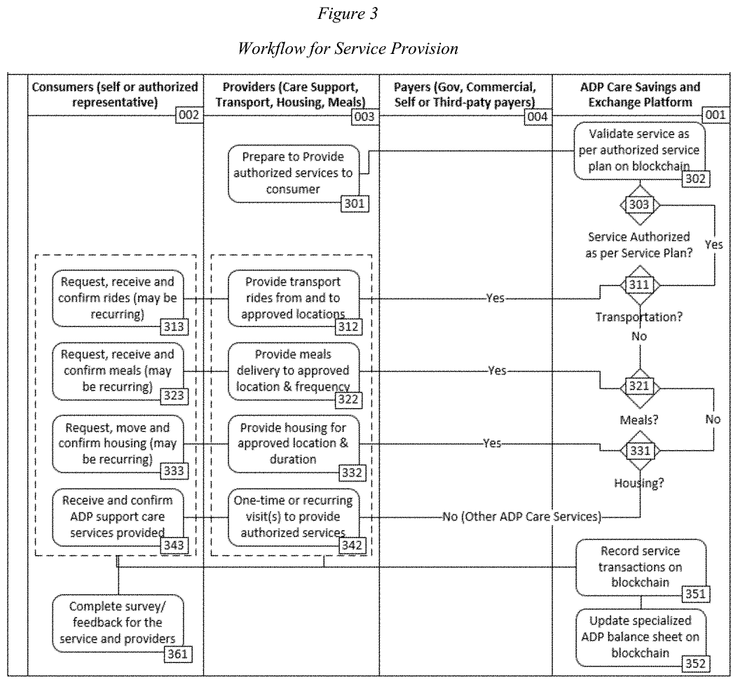

This plan is then used by the selected providers to schedule ADP services including daily living support, transport, meal and housing services 241. The consumer may review and accept the schedule or suggest changes, if any 242. At the end of this intelligent service planning workflow, the service schedule is finalized and providers are prepared to provide the services requested. Referring to FIG. 3, the overall workflow for service provision or delivery through the ADP services exchange 001 is depicted with primary involvement of consumers 002 and providers 003. Aligned with the service schedule defined during the intelligent service planning, the service provider is prepared to provide services to the consumer 101. The exchange 001 validates the services and providers against the authorized services and providers stored on blockchain solution 302. If the service is authorized 303, the provider can continue to provide service 303. By way of example, if the service is transportation 311, then the service renderer is a transportation provider (e.g. Lyft driver) who provides rides from and to the approved locations on the plan 312. The consumer may submit requests for a ride or it may be automatically scheduled as per the plan. The consumer receives and confirms the receipt of the transportation rides that may be onetime or recurring 313. Similar to transportation, ADP service providers 003 may provide meal delivery 322, housing 332 and another support service 342. Consumers receive and confirm the meals when delivered including recurring meal delivery, if authorized 323. For the housing services, a consumer may move and occupy the authorized housing for pre-defined duration and confirm the stay onetime or recurring basis as needed per the authorization 333. For all other ADP care support services, a consumer may receive those services and confirm receipt of the services for tracking purpose 342. The exchange 001 records all completed services on the blockchain 105 to allow partner systems to access those and reconcile with their own tracking, if any. The consumer ADP balance sheets are also kept up to date regarding the recording of assets and liabilities 352. Lastly, consumers have the option to complete a survey or provide feedback about the services and providers 361 to capture their satisfaction as well as review/rating for the provider. Consumers may receive alerts via the exchange, for example, that the provider service has been scheduled, or that payment has been completed for the provider service.

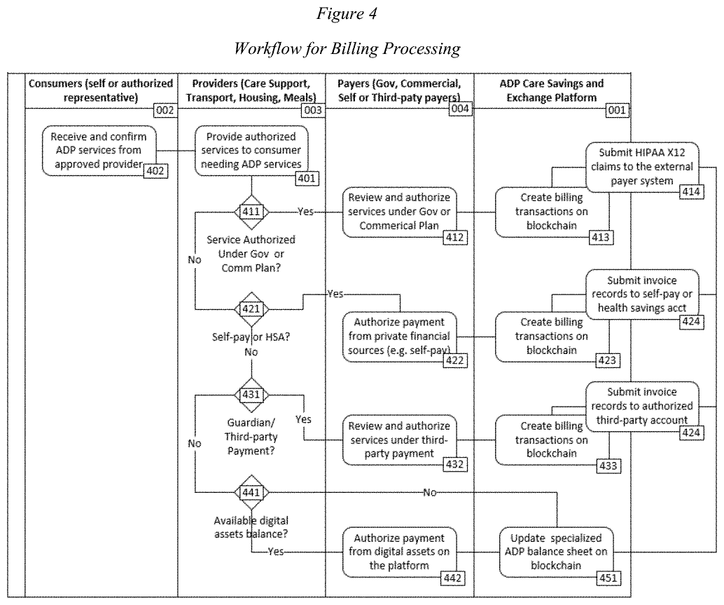

Referring to FIG. 4, the overall workflow for billing through the ADP services exchange 001 is depicted with primary involvement of providers 003 and payers 004 (including consumers 002 acting as payers 004 themselves for the self-pay). This is a continuation of the workflow after the service delivery is completed. ADP service providers deliver the service(s) to the consumer needing the services 401 and the consumer receives and confirms the service provided 402. If the service was authorized under a government or commercial plan 411, then the payer or authorized representative of the payer reviews and approves the services 412. This review and approval can also be configured to automate the process so that payer does not have review each and every service transaction. The exchange 001 then generates automated billing transactions or claims on the blockchain as immutable transactions 413 to support billing and payment as well as recording and tracking every transaction. Since these payers typically work with the Health Insurance Portability and Accountability Act (HIPAA) X12 claims, the exchange generates the claims in these standard transaction formats and submits them to the external payer system 414 on behalf of the provider. It is contemplated that a person of ordinary skill would understand that other types of claims may be utilized.

If services are approved under self-pay or Health Savings Account (HSA) 421, then consumers 002 themselves act as payers 004 to authorize the payments from the financial sources 422. This review and payment authorization is also configured to automate the process if the payer does not have to authorize individual service transaction. The exchange 001 then generates automated billing transactions or claims on the blockchain as immutable transactions 413 to support the billing and payment as well as the recording and tracking of the transactions. The exchange then submits the invoice to the self-pay payment sources 424 on behalf of the provider. If services are authorized under guardian or third-party sources 431, then a similar process is followed for approval 432, creation of billing transactions 433 and submitting invoice for payment 434. Lastly, when service is to be paid by available digital assets on the account 441, then payer authorizes payment 442. The exchange 001 also updates the balance sheet for all of the claims/invoices and payment transactions 451.

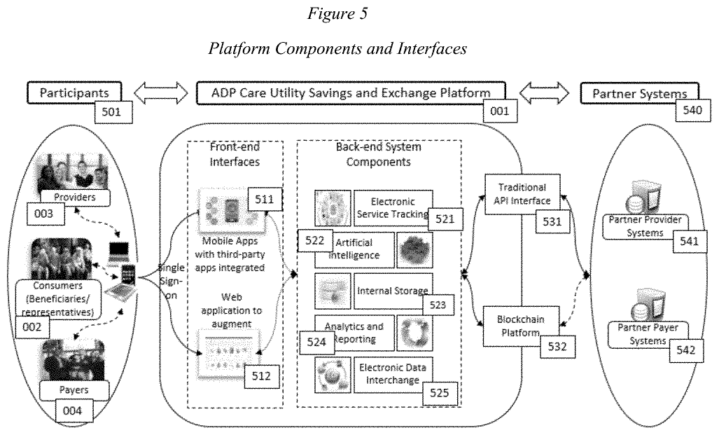

Referring to FIG. 5, the overview of the various interfaces of the ADP services exchange 001 is presented. Various types of users, collectively referred as participants 501, including consumers (beneficiaries or their authorized representatives) 002, providers 003 and payers 004 rely on the proposed ADP services exchange 001. Similarly, external partner systems 540 interact with the exchange 001 to facility electronic information exchange and seamless integration of processes. The exchange leverages the speed, security, and agility of health interoperability standards and blockchain smart contracting capabilities, resulting in a sustainable exchange that can be adapted wherever and whenever a service is required. This exchange delivers measurable business value to the consumers, payers and providers as described below. The exchange includes the following distinct capabilities as described below.
a. Mobile apps: Mobile applications 511 are used to connect the exchange 001 and the primary user interface for the participants 501. In some embodiments, there are three separate apps for different user groups: consumers 002, providers 003 and payers 004. Separate apps enables design and customization of the app interface specifically for the user groups so they can interact with the platform easily.
b. Web applications: While mobile apps 511 can provide all functionalities, there is often a need to access the functionality without relying on mobile app and connectivity. The exchange provides a web application 512 as an alternate interface for all participants 501 to interact with the exchange 001. Most functionality that can be accessed through mobile apps 511 can also be accessed via web application 512 except some of the functions that rely on mobile platform and smartphone capabilities such as capturing and tracking user's location through a global position system GPS, or any other applicable geolocation technology available.
c. Electronic Service Tracking (EST): This feature enables electronic tracking for the scheduling and effective delivery of the services needed for the ADP care. This include tracking of transportation rides, meal delivery, housing and other home and community based services.
d. Artificial Intelligence Engine (AI Engine): The AI engine provides a backbone for all AI related functions in the exchange including prediction of needs based on the assessment data, recommendations of services and providers, normalization of the digital assets based on multiple attributes.
e. Internal Storage: The internal storage 523 is the master database for all information collected through mobile apps 511 or web application 512 or processed in the exchange 001 through service tracking 521, AI 522, analytics 524 or Electronic Data Interchange (EDI) 525 modules.
f. Analytics and Reporting: This feature provides data processing and analytics capabilities 524 for more visualization, reporting and decision support. The results from this are available to the user groups as appropriate to help them enhance timeliness and reliability of service delivery.
g. Electronic Data Interchange Engine: This EDI engine 525 generates the data files in required format for sending to the partner systems 540 or processes the data received from the partner systems 540 to facilitate electronic exchange of the data seamlessly. This includes but is not limited to service delivery transactions and claims exchange with the provider systems 541 and eligibility information, pre-authorization, claims, payments with the payer systems 542.
h. Traditional Application Program Interface (API): This API interface 531 provides open RESTful API for the partner systems 540 to interact with the exchange using traditional integration method when they are not prepared to interact through the blockchain platform. It is understood that other APIs may be utilized as understood by a person of ordinary skill in the art.
i. Blockchain Platform: This Blockchain platform 532 is a Hyperledger Fabric based blockchain enabled solution that provides a secured, distributed and immutable network to record and exchange transactions between the exchange 001 and partner systems 540 that are prepared to connect to a blockchain. It also provides smart contracts using Hyperledger chaincode to validate the transactions before they are recorded on the blockchain network as well as the capability to create and maintain cryptographic digital tokens as digital assets as depicted herein.
j. Partner Systems: As described above, the participants 501 include providers 003 and payers 004 that access the exchange 001. While a single set of interfaces makes sense for ensuring all users are accessing the exchange the same way, it is often not an ideal approach. Many large organization partners have their own systems i.e. provider systems 541 and payer systems 542 for managing their workflow, data collection and reporting. So the exchange 001 integrates with these partner systems 540 through traditional API interface 531 or blockchain interface 532 as described above.

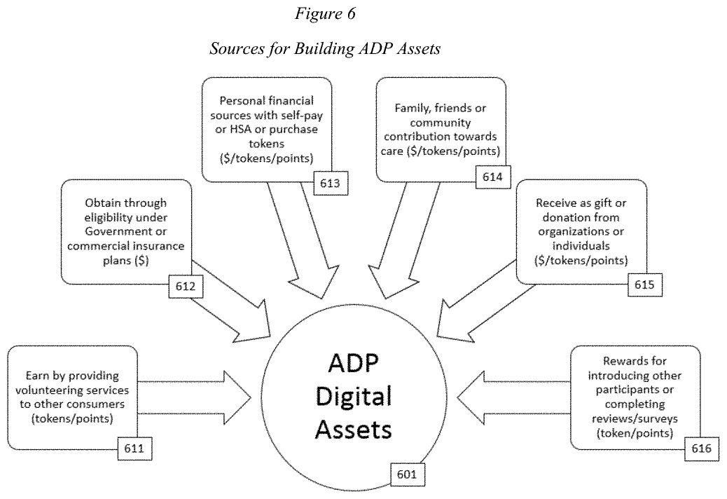

Referring to FIG. 6, the exchange provides for various sources to build ADP digital assets 601 in the exchange. As described earlier in reference to FIG. 1, the exchange provides multiple ways for consumers to acquire the necessary assets to support their ADP services needs in the present or over their lifetime. These methods of acquiring assets including digital ADP assets (cryptographic digital tokens or reward points) depend on various factors such as eligibility, savings, income, relationships with other participating organizations and individuals as described below. Consumers may receive alerts via the exchange, for example, of the balance of or an increase/decrease of, the digital tokens or rewards.
a) Earn by providing volunteering services to other consumers 611: A consumer can volunteer to provide services through participating organizations such as Community Based Organizations (CBO) or directly to participating individual consumers. Such volunteering providers (who may also be consumers) can earn the digital tokens or rewards points into their ADP accounts based on the services provided, time spent and other factors that help determine the value of the volunteered time.
b) Obtain through eligibility under government or commercial insurance plans 612—Consumers that were eligible for commercial plans or government support plans can build their assets through the service plan authorized by the insurance company or government agency, to build equity in the approved services dictated by the plan(s) for which they are eligible.
c) Personal financial sources with self-pay or HSA or purchase digital tokens/points 613—If a consumer does not have digital tokens/points or eligibility for the support plans, they may be able to support their services directly through self-pay or healthcare spending account to help build the assets. Consumers can also use their personal savings to purchase digital tokens and rewards points from the participating organizations or individuals. Purchasing digital tokens is similar to self-pay option except that digital tokens may just be used to build assets for future use versus self-pay or HSA are used to pay for services in present.
d) Family, friends or community contribution towards care 614—When consumer does not have own financial sources, then financial contributions or digital tokens/points from family, friends or community may be used by consumers to pay for the services directly.
e) Receive as gift or donation from organizations or individuals 615—The consumers that do not have sufficient assets described above to support their services can search and request for digital assets as gift or donation from other organizations or individuals on the exchange as charity contribution.
f) Rewards for introducing other participants or completing reviews/surveys 616—Consumers can earn digital tokens or points by introducing other individuals or organizations to the exchange. Specific criteria may be applied to determine number of digital tokens or points awarded for the building the network from the set-aside quota digital tokens or points. As the network expands, value will likely go up and hence fewer digital tokens/points will be awarded for these introductions. As Rewards may also be offered for review or completion of surveys.

Referring to FIG. 7, the exchange provides for the normalization of ADP balance sheets 701 using attributes associated with the digital assets and liabilities. Exchange of services for digital tokens or rewards points requires reliable and precise valuation of the services. The exchange uses AI processing to normalize valuation of ADP digital assets (cryptographic digital tokens or reward points) and liabilities (services needed) based on the following attributes:
a) What 711—This is an objective measure based on what services were provided (e.g. volunteering) to acquire assets and which services are to be provided using those assets. This is typically characterized by the standardized service code when available or customized value set. For example, a consumer may provide support care services to earn digital tokens/reward points and then use those to receive meals service.
b) When 712—This is an objective attribute to associate time period when the assets are acquired and when they are traded in for the services in present (at current value) or future (extrapolated or predicted) value for the services depending on when the consumer wants to receive services.
c) Where 713—This very important objective attribute is associated with the normalization of the assets based on where they are acquired and where they will be spent. This attribute has two-dimension location that may go across local community, county, state, country or even wider geo-political boundaries. For example, a consumer may acquire assets by contributing volunteer time in the United States but then use those assets to receive home care services in another country where the valuation allows more care services for the same assets as compared to the United States. Or the consumer could have family members that live outside of the consumer's country who would be able to provide support and best meet the consumer's needs.
d) Who 714—This is more of a subjective attribute for the valuation of assets using type and settings of provider as well as a valuation of relationship with consumers. For instance, a consumer may have a more subjective valuation of care provided by someone within their community then by someone outside of their community, or someone with whom they have an existing and trusted relationship, rather than someone they do not know.

There are several complex rules involved in normalization for the token valuation. For example, when service is to be provided in different geographical region say another country, normalization technique and rules take into account various factors, for example, the currency conversion ratio, cost of living differences, difference in average cost of service provision, and availability of the providers including volunteers in the region. Once the exchange normalizes the assets and liabilities for different region, the exchange displays a geographical map of all regions that payers and consumers can use to determine how they can benefit for their digital tokens used in another geographical region. This helps them make decision to move to different location to receive best value for their assets.

The normalized valuation as described above allows the determination of the value/balance of the organization or individual accounts. The exchange maintains a specialized ADP balance sheet that records ADP assets as described above and current or life-time ADP liabilities needs projected/predicted against those assets.

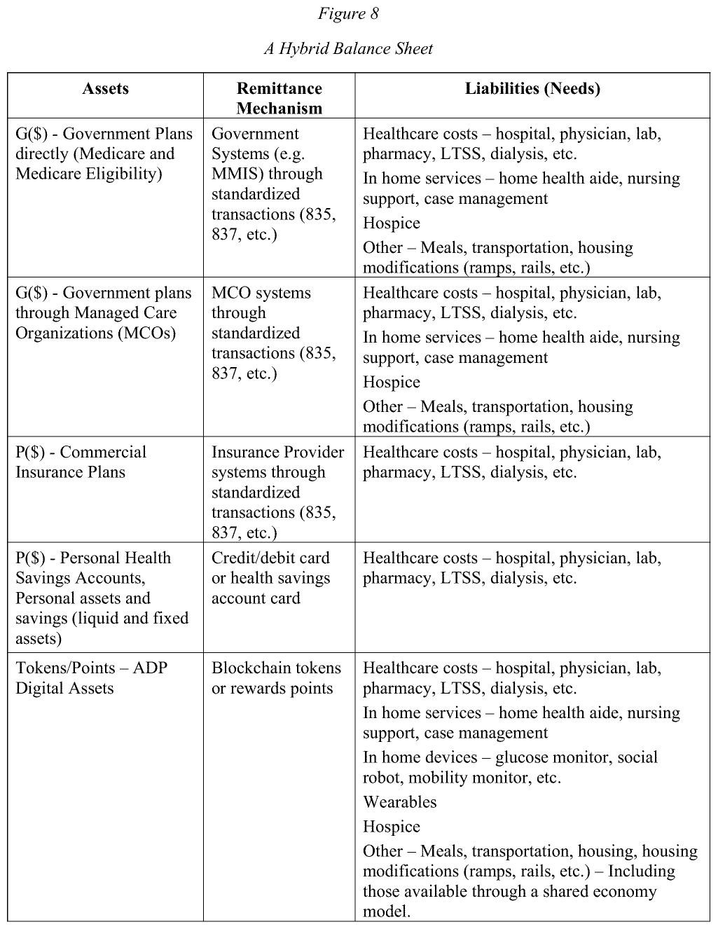

Referring to FIG. 8, various components of ADP balance sheet are depicted showing the ability to combine traditional methods and innovative approaches to address a consumer's liabilities that occur during ADP phases of life. ADP balance sheets with normalized assets and liabilities allow clear indication of gaps or surplus of coverage for ADP support services, at a government program, State, county, community, family or consumer level to any authorized participant. Within the first column of FIG. 8, the first two categories of assets are administered by government jurisdictions such as counties, states, the federal government, or through government agents. Operating and financial costs are high; at times 25% or more—such as in the case of high cost Medicaid Waiver or Managed Care Organization (MCO) programs, where there is both federal and state administrative overhead in addition to the approximately 15% administrative costs of the MCO—which doesn't take into account the provider administrative costs.

The third category of assets are commercial health plans which generally operated between 12-18% administrative overhead; which again doesn't take into account the overhead built into the provider payment. The fourth category of assets are administered by the consumer (or family member of the consumer). Consumers can purchase more services per dollar than government program are able to, and there are much fewer restrictions related to funds utilization. The fifth category of assets are the digital tokens and reward points. The exchange fully supports this category by allowing individuals to contribute, save, donate, exchange, and utilize ADP assets across boundaries of time, type, person and location while avoiding most of the costly overhead associated with other methodologies.

The second column, Remittance Mechanism, describes the administrative mechanisms that are utilized for each category of assets. They range from standardized transactions, to electronic card systems to the exchange of digital tokens.

The third column lists the categories of liabilities a consumer might face while disabled or aged. The exchange can address all categories of potential liabilities, such as in-home services—(home health aide, nursing support, case management), in-home devices—(glucose monitor, social robot, mobility monitor, etc.), wearables, and other services such as meals, transportation, housing, housing modifications (such as ramps and rails)—including those which are available through a shared economy model. Many of these are not supported by any other model today. The exchange makes use of financial technology for banking and transactions on an immutable, distributed and permissioned blockchain network. This includes use of cryptographic digital tokens for banking and valuation of ADP digital assets as well as use of cryptographic digital tokens as an acceptable payment option for the services.

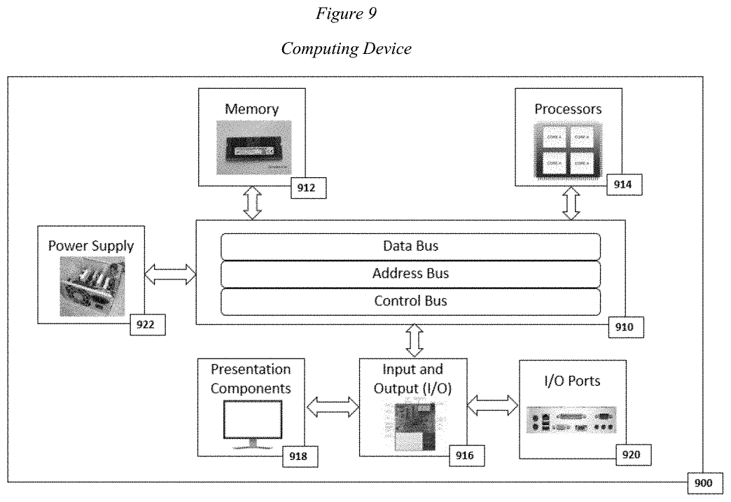

Referring to FIG. 9, a computing device 900 includes a bus 910 that directly or indirectly couples the following devices: memory 912, one or more processors 914, input/output (I/O) components 916, one or more presentation components 918, I/O ports 920, and a power supply 922. This is an exemplary computing device that can be used in connection with the embodiments of this invention. Computing devices may include a desktop computer, a laptop or handheld device. It may also refer to a server, whether a physical server for use as a web or database server, or a virtual server, such as may reside in a virtual environment or cloud.

It is believed that the present invention and many of its attendant advantages will be understood by the forgoing description. It is also believed that it will be apparent that various changes may be made in the form, construction and arrangement of the components thereof without departing from the scope and spirit of the invention or without sacrificing all of its material advantages. The form herein before described being merely an explanatory embodiment thereof. It is the intention of the following claims to encompass and include such changes.

What is claimed is:

1. A system for acquisition and payment of aging, disability, and palliative (ADP) services, comprising at least one computing device configured to:
register at least one consumer by storing said consumer's consumer information, including said consumer's name and other identifying information and creating a consumer account for said consumer from said consumer information;
receive from said consumer a consumer itemization of said consumer's assets and liabilities;
create and store a consumer ADP balance sheet containing said consumer itemization in the consumer account;
create estimates of needs and liabilities of said consumer over a future period of time and store the estimates in the consumer ADP balance sheet;
acquire a digital token for purchasing ADP services;
update the consumer ADP balance sheet to reflect the digital token acquired;
register at least one service provider by storing said service provider's provider information, including said at least one service provider's name and other identifying information in a provider account;
create and store a provider ADP balance sheet of said service provider in said provider account;
select a service provider and estimate a value for ADP services to be provided to the consumer;
procure ADP services from a selected service provider;
make payment for provided ADP services in an amount of digital tokens from the consumer account; and
update the consumer ADP balance sheet and the provider ADP balance sheet to reflect the payment.

2. The system of claim 1, wherein said digital tokens, the consumer ADP balance sheet, and the provider ADP balance sheet are stored in a blockchain.

3. The system of claim 1, wherein said digital tokens are acquired by said consumer performing volunteer work in exchange for the digital tokens.

4. The system of claim 1, wherein at least one third party makes a donation of digital tokens to the consumer account.

5. The system of claim 1, wherein digital tokens are earned by said consumer in accordance with actions of said consumer in response to requests via the ADP services exchange.

6. The system of claim 1, wherein the ADP services exchange transmits an alert to said consumer upon a change in the amount of digital tokens in the consumer account.

7. The system of claim 1, wherein said at least one computing device is further configured to:
register at least one payer by entering said payer information, including the payer name and other identifying information in a payer account;
create and store a payer ADP balance sheet in said payer account;
receive a request from at least one consumer to procure digital tokens from at least one payer;
transfer digital tokens from the payer account to the consumer account; and
update the consumer ADP balance sheet and payer ADP balance sheet to reflect the transfer.

8. The system of claim 1, wherein the amount of digital tokens required for said payment for the ADP services is normalized based upon service type, service level, location and time.

9. The system of claim 8, wherein the normalization of the amount of digital tokens is performed via an artificial intelligence engine.

10. The system of claim 1, wherein at least one service provider is recommended to said at least one consumer via the ADP services exchange for the service desired by said consumer, based upon said consumer's past experience with service providers, reviews and ratings by other consumers, location, and availability.

11. A method for acquisition and payment of aging, disability, and palliative (ADP) services, comprising:
registering, via a computing device, at least one consumer by storing said consumer's consumer information, including said consumer's name and other identifying information and creating a consumer account from said consumer's information;
receiving, via the computing device, from said consumer a consumer itemization of the consumer's assets and liabilities;
creating and storing a consumer ADP balance sheet containing said consumer itemization in the consumer account;
creating estimates of needs and liabilities of said consumer over a future period of time and storing the estimates in the consumer ADP balance sheet;
acquiring a digital token for purchase of ADP services;
updating the consumer ADP balance sheet to include said digital token acquired;
registering, via a computing device, at least one service provider by storing said service provider's provider information, including name and other identifying information, in a service provider account;
creating and storing a provider ADP balance sheet in said provider account;
selecting at least one service provider and estimating a value for ADP services to be provided to said consumer;
procuring ADP services from at least one service provider;
making payment for provided ADP services in an amount of digital tokens from the consumer account; and
updating the consumer ADP balance sheet and provider ADP balance sheet to reflect the payment.

12. The method of claim 11, wherein said consumer accesses and registers via ADP services exchange via an application installed on said consumer's computing device.

13. The method of claim 11, wherein said digital tokens, the consumer ADP balance sheet, and the provider ADP balance sheet are stored in a blockchain.

14. The method of claim 11, wherein the consumer acquires said digital tokens for performing volunteer work.

15. The method of claim 11, wherein a third party makes a donation of digital tokens to the consumer account.

16. The method of claim 12, wherein the ADP services exchange grants digital tokens to said consumer account.

17. The method of claim 11, further comprising:
registering, via a computing device, at least one a payer by storing said payer information, including the payer's name and other identifying information and creating a payer account from said payer information;
creating and storing an ADP balance sheet of said payer in said payer account;
receiving, via a computer device, request from the consumer to procure digital tokens from said payer;
receiving digital tokens from the payer; and
updating the payer ADP balance sheet and the consumer payer sheet to reflect an exchange of digital tokens.

18. The method of claim 12, wherein the ADP services exchange transmits an alert to said consumer upon a change in the amount of digital tokens in said consumer account.

19. The method of claim 11, wherein the amount of digital tokens required for the payment for the ADP services is normalized based upon service type, service level, location and time.

20. The method of claim 19, wherein normalization of the amount of digital tokens is performed via an artificial intelligence engine.

21. The method of claim 12, wherein at least one service provider is recommended to the consumer via the ADP services exchange for a service desired by said consumer, based upon said consumer's past experience with service providers, reviews and ratings by other consumers, location, and availability.
